(19) United States
(12) Patent Application Publication
(10) Pub. No.: US 2026/0233209 A1
(43) Pub. Date: Aug. 13, 2026

(54) CRACKING ALKYLENE GLYCOL DIALKYL ETHER

(71) Applicant: DOW GLOBAL TECHNOLOGIES LLC, Midland, MI (US)

(72) Inventors: Kimberly Tam Dinh, Midland, MI (US); Wen-Sheng Lee, Midland, MI (US); Sung-Yu Ku, Manvel, TX (US)

(21) Appl. No.: 19/153,653

(22) PCT Filed: Feb. 15, 2024
(86) PCT No.: PCT/US2024/015966

§ 371 Date: Aug. 5, 2025

Related U.S. Application Data
(60) Provisional application No. 63/446,915, filed on Feb. 20, 2023.

(51) Int. Cl. B01J 29/70; B01J 29/08; C07C 41/26; C07C 43/13

(52) U.S. Cl. CPC B01J 29/7007; B01J 29/088; C07C 41/26; C07C 43/13

(57) ABSTRACT

A method of generating an alkylene glycol monoalkyl ether includes the steps of contacting a metallosilicate catalyst with a reaction composition comprising 5 wt % or greater of alkylene glycol dialkyl ether based on the total weight of the reaction composition; and generating the alkylene glycol monoalkyl ether from the alkylene glycol dialkyl ether of the reaction composition.

### BACKGROUND

### Field of the Invention

The present disclosure generally relates to alkylene glycol dialkyl ether, and more specifically to methods of cracking the alkylene glycol dialkyl ether to form alkylene glycol monoalkyl ether.

### Introduction

Production of secondary alcohol ethoxylate surfactants can be carried out by the catalyzed ethoxylation of (poly)alkylene glycol monoalkyl ether (“monoalkyl ether”). The monoalkyl ether is formed from an olefin and a (poly)alkylene glycol via an etherification reaction using metallosilicate catalysts. The etherification reaction also produces (poly)alkylene glycol secondary dialkyl ether (“dialkyl ether”). The selectivity of a catalyst or a reaction indicates the relative proportions of reaction products produced. Typically, a high selectivity of the desired reaction product is advantageous in maximizing the output of the reaction. Metallosilicate catalysts offer a selectivity for monoalkyl ether of greater than 80% which is advantageous as dialkyl ether is a byproduct of the reaction which results in lower raw material utilization rate. Additionally, dialkyl ether is deleterious to properties of the secondary alcohol ethoxylate surfactants if dialkyl ether does not separate from monoalkyl ether and is carried into alkoxylation.

Attempts at addressing the formation of dialkyl ether have been attempted. For example, U.S. Pat. No. 6,417,408 (“the '408 patent”) discloses a process for producing a (poly)alkylene glycol monoalkyl ether by reacting an olefin and a (poly)alkylene glycol in the presence of a catalyst. The '408 patent explains that during the reaction to produce monoalkyl ether, a specific reaction equilibrium exists in the reaction medium between the amount of monoalkyl ether and the combined amount of polyalkylene glycol and dialkyl ether. As a result of this understanding, the '408 patent “found that when the olefin and the (poly)alkylene glycol are reacted to produce the (poly)alkylene glycol monoalkyl ether, it is effective to add the (poly)alkylene glycol dialkyl ether to inhibit the formation thereof as well.” Put another way, by recycling the dialkyl ether back into the reaction, the equilibrium could be artificially shifted to reduce the production of dialkyl ether and thereby increase the monoether yield. Regardless, the production and lack of use of dialkyl ether results in overall lower monoalkyl ether yield in the reaction system.

Attempts at using or reacting dialkyl ethers have been attempted. For example, U.S. Pat. No. 4,368,337 discloses the cracking of di-tertiary alkyl ethers by reacting a feed glycol di-tertiary alkyl ether with water using a strongly acidic cation-exchange resin as a catalyst. Di-tertiary alkyl ether can form a stable tert-alkyl cation under acidic conditions which makes the cracking process easier than secondary alkyl ethers. The lack of prior attempts at cracking secondary alkyl ethers and primary alkyl ethers indicate the substantial and unresolved challenges posed by the cracking of secondary and primary dialkyl ethers.

The inventors of the present application have discovered a process that effectively cracks the secondary dialkyl ether byproduct to the desired monoalkyl ether.

### SUMMARY OF THE DISCLOSURE

The inventors of the present application have discovered a process that effectively cracks the secondary dialkyl ether byproduct to the desired monoalkyl ether.

The invention is the result of discovering that while metallosilicate catalysts have traditionally been used to form the monoalkyl ether, the same metallosilicate catalysts can be used to convert the unwanted dialkyl ether directly into monoalkyl ether as well. The metallosilicate catalyst can be used to crack dialkyl ether either on its own, or in conjunction with a glycol and/or solvent. Specifically, a reaction composition having 5 wt % or greater of dialkyl ether can be used as a feedstock to form monoalkyl ether. The dialkyl ether may be sourced from previous reactions forming monoalkyl ether such that the overall monoalkyl ether yield of an entire reaction system is increased.

According to a first feature of the disclosure, a method of generating an alkylene glycol monoalkyl ether, comprises the steps of: contacting a metallosilicate catalyst with a reaction composition comprising 5 wt % or greater of alkylene glycol dialkyl ether based on the total weight of the reaction composition; and generating the alkylene glycol monoalkyl ether from the alkylene glycol dialkyl ether of the reaction composition.

According to a second feature of the present disclosure, the metallosilicate catalyst is an aluminosilicate compound having a crystal lattice that has had one or more metal elements substituted in the crystal lattice for a silicon atom.

According to a third feature of the present disclosure, the reaction composition comprises 50 wt % or greater of the alkylene glycol dialkyl ether based on the total weight of the reaction composition.

According to a fourth feature of the present disclosure, the reaction composition comprises 80 wt % or greater of the alkylene glycol dialkyl ether based on the total weight of the reaction composition.

According to a fifth feature of the present disclosure, the reaction composition comprises a glycol selected from the group consisting of monoethylene glycol, diethylene glycol, glycerol or combinations thereof.

According to a sixth feature of the present disclosure, the reaction composition comprises from 10 wt % to 80 wt % of the glycol based on the total weight of the reaction composition.

According to a seventh feature of the present disclosure, the reaction composition further comprises a solvent selected from the group consisting of an oxygenated solvent, a non-oxygenated solvent and combinations thereof.

According to an eighth feature of the present disclosure, the solvent is from 1 wt % to 80 wt % of the total weight of the reaction composition.

According to a nineth feature of the present disclosure, the solvent comprises both the oxygenated solvent and the non-oxygenated solvent.

According to a tenth feature of the present disclosure, the solvent comprises from 40 wt % to 60 wt % of the non-oxygenated solvent based on a total weight of the solvent.

### DETAILED DESCRIPTION

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

All ranges include endpoints unless otherwise stated.

Test methods refer to the most recent test method as of the priority date of this document unless a date is indicated with the test method number as a hyphenated two-digit number. References to test methods contain both a reference to the testing society and the test method number. Test method organizations are referenced by one of the following abbreviations: ASTM refers to ASTM International (formerly known as American Society for Testing and Materials); EN refers to European Norm; DIN refers to Deutsches Institut für Normung; and ISO refers to International Organization for Standards.

IUPAC codes describing Crystal structures as delineated by the Structure Commission of the International Zeolite Association refer to the most recent designation as of the priority date of this document unless otherwise indicated.

As used herein, the term weight percent (“wt %”) designates the percentage by weight a component is of a total weight of an indicated composition.

As used herein, a “CAS number” is the chemical services registry number assigned by the Chemical Abstracts Service.

### Method

The method of the present invention is directed to the generation of an alkylene glycol monoalkyl ether. The method may comprise steps of (a) contacting a metallosilicate catalyst with a reaction composition comprising 5 wt % or greater of alkylene glycol dialkyl ether based on the total weight of the reaction composition and (b) generating the alkylene glycol monoalkyl ether from the reaction composition.

### Metallosilicate Catalyst

As used herein the term “metallosilicate catalyst” is an aluminosilicate (commonly referred to as a zeolite) compound having a crystal lattice that has had one or more metal elements substituted in the crystal lattice for a silicon atom. The crystal lattice of the metallosilicate catalyst form cavities and channels inside where cations, water and/or small molecules may reside. The substitute metal element may include one or more metals selected from the group consisting of B, Al, Ga, In, Ge, Sn, P, As, Sb, Sc, Y, La, Ti, Zr, V, Cr, Mn, Pb, Pd, Pt, Au, Fe, Co, Ni, Cu, Zn. The metallosilicate catalyst may be substantially free of Hf. According to various examples, the metallosilicate may have a silica to alumina molar ratio of from 5:1 to 1,500:1 as measured using Neutron Activation Analysis. The silica to alumina molar ratio may be from 5:1 to 1,500:1, or from 10:1 to 500:1, or from 10:1 to 400:1, or from 10:1 to 300:1 or from 10:1 to 200:1. Such a silica to alumina molar ratio may be advantageous in providing a metallosilicate catalyst with an appropriate hydrophobic selectivity that adsorb non-polar organic molecules.

The metallosilicate catalyst may have one or more ion-exchangeable cations outside the crystal lattice. The ion-exchangeable cation may include H⁺, Li⁺, Na⁺, Rb⁺, Cs⁺, Mg²⁺, Ca²⁺, Sr²⁺, Ba²⁺, Sc³⁺, Y³⁺, La³⁺, R₄N⁺, R₄P⁺ (where R is H or alkyl).

The metallosilicate catalyst may take a variety of crystal structures. Specific examples of the metallosilicate catalyst structures include MFI (e.g. ZSM-5), MEL (e.g. ZSM-11), BEA (e.g. β-type zeolite), FAU (e.g. Y-type zeolite), MOR (e.g. Mordenite), MTW (e.g. ZSM-12), and LTL (e.g. Linde L), as described using IUPAC codes in accordance with nomenclature by the Structure Commission of the International Zeolite Association.

The crystalline frameworks of metallosilicate catalyst are represented by networks of molecular-sized channels and cages comprised of corner-shared tetrahedral [TO₄] (T=Si or Al) primary building blocks. A negative charge can be introduced onto the framework via the isomorphous substitution of a framework tetravalent silicon by a trivalent metal (e.g., aluminum) atom. The overall charge neutrality is then achieved by the introduction of cationic species compensating for the resulting negative lattice charge. When such a charge-compensation is provided by protons, Brønsted acid sites are formed rendering the resulting H-forms of zeolites strong solid Brønsted acids.

The metallosilicate catalysts may be used in the method in a variety of forms. For example, the metallosilicate catalysts may be powdered (e.g., particles having a longest linear dimension of less than 100 micrometers), granular (e.g., particles having a longest linear dimension of 100 micrometers or greater), or molded articles (e.g., pellets or extrudates) of powdered and/or granular metallosilicate catalysts.

The metallosilicate catalysts may have a surface area of 100 m²/g or greater, or 200 m²/g or greater, or 300 m²/g or greater, or 400 m²/g or greater, or 500 m²/g or greater, or 600 m²/g or greater, or 700 m²/g or greater, or 800 m²/g or greater, or 900 m²/g or greater, while at the same time, 1000 m²/g or less, or 900 m²/g or less, or 800 m²/g or less, or 700 m²/g or less, or 600 m²/g or less, or 500 m²/g or less, or 400 m²/g or less, or 300 m²/g or less, or 200 m²/g or less. Surface area is measured according to ASTM D4365-19.

Metallosilicate catalysts can be synthesized by hydrothermal synthesis methods. For example, the metallosilicate catalysts can be synthesized from heating a composition comprising a silica source (e.g., silica sol, silica gel, and alkoxysilanes), a metal source (e.g., metal sulfates, metal oxides, metal halides, etc.), and a quaternary ammonium salt such as a tetraethylammonium salt or tetrapropylammonium to a temperature of about 100° C. to about 175° C. until a crystal solid forms. The resulting crystal solid is then filtered off, washed with water, and dried, and then calcined at a temperature form 350° C. to 600° C.

Examples of suitable commercially available, metallosilicate catalysts include CP814E, CP814C, CP811EL, CP811C-300, CBV 712, CBV 720, CBV 760, CBV 2314, CBV 10A from ZEOLYST INTERNATIONAL™ of Conshohocken, PA.

### Reaction Composition

The reaction composition is contacted with the metallosilicate catalyst to produce monoalkyl ether. The reaction composition comprises the dialkyl ether and optionally one or more of a glycol and a solvent.

### Dialkyl Ether

The dialkyl ether is present in the reaction composition and used as a feed source for generating the monoalkyl ether. Specifically, the dialkyl ether is cracked into monoalkyl ether. As used herein, the terms “crack” or “cracked” indicate that a molecular structure is reduced in size into one or more moieties. The dialkyl ether can be the product of one or more previous reactions. For example, the dialkyl ether can be produced by a method comprising the steps of (1) contacting an olefin, a glycol, a metallosilicate catalyst and (2) generating an alkylene glycol monoalkyl ether and the alkylene glycol dialkyl ether. The olefin used in the method may be linear, branched, acyclic, cyclic, or mixtures thereof. The olefin may have from 5 carbons to 30 carbons (i.e., C₅-C₃₀). The olefin may include alkenes such as alpha (α) olefins, internal disubstituted olefins, or cyclic structures (e.g., C₃-C₁₂ cycloalkene).

The dialkyl ether is described by Structure (I)

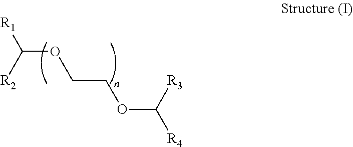

wherein R₁, R₂, R₃, and R₄ are alkyls and n has a value of 1 to 3. Each of the alkyls of R₁-R₄ may independently have from one carbon atom (i.e., be a C₁ alkyl) to sixteen carbon atoms (i.e., be a C₁₆ alkyl). For example, each alkyl may independently be a C₁ alkyl, or a C₂ alkyl, or a C₃ alkyl, or a C₄ alkyl, or a C₅ alkyl, or a C₆ alkyl, or a C₇ alkyl, or a Cs alkyl, or a C₉ alkyl, or a C₁₀ alkyl, or a C₁₁ alkyl, or a C₁₂ alkyl, or a C₁₃ alkyl, or a C₁₄ alkyl, or a C₁₅ alkyl, or a C₁₆ alkyl. The alkyl of R₁ may be saturated or unsaturated. The sum of carbon atoms present in R₁ and R₂ may be from 7 to 17. For example, the sum of carbon atoms present in R₁ and R₂ may be 7 or greater, or 8 or greater, or 9 or greater, or 10 or greater, or 11 or greater, or 12 or greater, or 13 or greater, or 14 or greater, or 15 or greater, or 16 or greater, while at the same time, 17 or less, or 16 or less, or 15 or less, or 14 or less, or 13 or less, or 12 or less, or 11 or less, or 10 or less, or 9 or less, or 8 or less. The sum of carbon atoms for R₃ and R₄ may be any of the above-noted values for the sum of R₁ and R₂. The value n of Structure (I) has a value from 1 to 3. For example, n may be 1, 2, or 3. The number of carbons present in R₁-R₄ and the value of n are all determined according to ¹³C nuclear magnetic resonance characterization (“NMR”). In specific examples of the dialkyl ether, the variables of Structure (I) may be n=1, R₁+R₂=11 or R₁+R₂=13, and R₃+R₄=11 or R₃+R₄=13. The dialkyl ether is secondary dialkyl ether.

The reaction composition comprises 5 wt % or greater of the alkylene glycol dialkyl ether based on the total weight of the reaction composition. For example, the reaction composition may comprise 5 wt % or greater, or 10 wt % or greater, or 15 wt % or greater, or 20 wt % or greater, or 25 wt % or greater, or 30 wt % or greater, or 35 wt % or greater, or 40 wt % or greater, or 45 wt % or greater, or 50 wt % or greater, or 55 wt % or greater, or 60 wt % or greater, or 65 wt % or greater, or 70 wt % or greater, or 75 wt % or greater, or 80 wt % or greater, or 85 wt % or greater, or 90 wt % or greater, or 95 wt % or greater, while at the same time, 100 wt % or less, or 95 wt % or less, or 90 wt % or less, or 85 wt % or less, or 80 wt % or less, or 75 wt % or less, or 70 wt % or less, or 65 wt % or less, or 60 wt % or less, or 55 wt % or less, or 50 wt % or less, or 45 wt % or less, or 40 wt % or less, or 35 wt % or less, or 30 wt % or less, or 25 wt % or less, or 20 wt % or less, or 15 wt % or less, or 10 wt % or less of the dialkyl ether based on the total weight of the reaction composition.

### Glycol

As explained above, the reaction composition may comprise a glycol. The glycol may be useful in increasing the monoalkyl ether yield while cracking the dialkyl ether. The glycol utilized in the method comprises two or more hydroxyl groups. The glycol may include 1 carbon or greater, or 2 carbons or greater, or 3 carbons or greater, or 4 carbons or greater, or 5 carbons or greater, or 6 carbons or greater, or 7 carbons or greater, or 8 carbons or greater, or 9 carbons or greater, while at the same time, 10 carbons or less, or 9 carbons or less, or 8 carbons or less, or 7 carbons or less, or 6 carbons or less, or 5 carbons or less, or 4 carbons or less, or 3 carbons or less, or 2 carbons or less. The glycol may be selected from the group consisting of monoethylene glycol, diethylene glycol, propylene glycol, triethylene glycol, polyethylene glycol, monopropylene glycol, dipropylene glycol, tripropylene glycol, polypropylene glycol, 1,3-propanediol, 1,2-butanediol, 2,3-butanediol, 1,4-butanediol, 1,6-hexanediol, 1,4-cyclohexanemethanediol, glycerol and/or combinations thereof. According to various examples, the glycol is a (poly)alkylene glycol such as monoethylene glycol, diethylene glycol, propylene glycol and triethylene glycol.

The reaction composition may comprise from 0 wt % to 80 wt % of the glycol based on the total weight of the reaction composition. For example, the reaction composition may comprise 0 wt % or greater, or 5 wt % or greater, or 10 wt % or greater, or 15 wt % or greater, or 20 wt % or greater, or 25 wt % or greater, or 30 wt % or greater, or 35 wt % or greater, or 40 wt % or greater, or 45 wt % or greater, or 50 wt % or greater, or 55 wt % or greater, or 60 wt % or greater, or 65 wt % or greater, or 70 wt % or greater, or 75 wt % or greater, while at the same time, 80 wt % or less, or 75 wt % or less, or 70 wt % or less, or 65 wt % or less, or 60 wt % or less, or 55 wt % or less, or 50 wt % or less, or 45 wt % or less, or 40 wt % or less, or 35 wt % or less, or 30 wt % or less, or 25 wt % or less, or 20 wt % or less, or 15 wt % or less, or 10 wt % or less, or 5 wt % or less of the glycol based on the total weight of the reaction composition.

### Solvent

The reaction composition may comprise the solvent. The solvent may comprise an oxygenated solvent and/or a non-oxygenated solvent. As used herein, the term “oxygenated solvent” means a compound comprising carbon, hydrogen and oxygen atoms that solubilize one or more of the glycol, dialkyl ether and/or monoalkyl ether. As used herein, the term “non-oxygenated solvent” means a compound which does not include an oxygen atom and which solubilize one or more of the glycol, dialkyl ether and/or monoalkyl ether.

The solvent may be from 0 wt % to 95 wt % of the total weight of the reaction composition. For example, the solvent may be 0 wt % or greater, or 0.1 wt % or greater, or 0.5 wt % or greater, or 1 wt % or greater, or 5 wt % or greater, or 10 wt % or greater, or 15 wt % or greater, or 20 wt % or greater, or 25 wt % or greater, or 30 wt % or greater, or 35 wt % or greater, or 40 wt % or greater, or 45 wt % or greater, or 50 wt % or greater, or 55 wt % or greater, or 60 wt % or greater, or 65 wt % or greater, or 70 wt % or greater, or 75 wt % or greater, or 80 wt % or greater, or 85 wt % or greater, or 90 wt % or greater, while at the same time, 95 wt % or less, or 90 wt % or less, or 85 wt % or less, or 80 wt % or less, or 75 wt % or less, or 70 wt % or less, 65 wt % or less, or 60 wt % or less, 55 wt % or less, or 50 wt % or less, 45 wt % or less, or 40 wt % or less, 35 wt % or less, or 30 wt % or less, 25 wt % or less, or 20 wt % or less, 15 wt % or less, or 10 wt % or less, 5 wt % or less, or 1 wt % or less, or 0.5 wt % or less of the total weight of the reaction composition.

The solvent may comprise from 0 wt % to 100 wt % of the oxygenated solvent based on a total weight of the solvent and 0 wt % to 100 wt % of the non-oxygenated solvent based on a total weight of the solvent. For example, the solvent may comprise 0 wt % or greater, or 5 wt % or greater, or 10 wt % or greater, or 15 wt % or greater, 20 wt % or greater, or 25 wt % or greater, or 30 wt % or greater, or 35 wt % or greater, or 40 wt % or greater, or 45 wt % or greater, or 50 wt % or greater, or 55 wt % or greater, or 60 wt % or greater, or 65 wt % or greater, or 70 wt % or greater, or 75 wt % or greater, or 80 wt % or greater, or 85 wt % or greater, or 90 wt % or greater, or 95 wt % or greater, while at the same time, 100 wt % or less, or 95 wt % or less, or 90 wt % or less, or 85 wt % or less, or 80 wt % or less, or 75 wt % or less, or 70 wt % or less, or 65 wt % or less, or 60 wt % or less, or 55 wt % or less, or 50 wt % or less, or 45 wt % or less, or 40 wt % or less, or 35 wt % or less, or 30 wt % or less, or 25 wt % or less, or 20 wt % or less, or 15 wt % or less, or 10 wt % or less, or 5 wt % or less of the oxygenated solvent and/or the non-oxygenated solvent based on the total weight of the solvent.

The oxygenated solvent is selected from the group consisting of cresols, o-cresol, m-cresol, p-cresol, phenol, guaiacol, methyl benzoate, dimethyl phthalate, diglyme, triglyme, diethyl phthalate, dibutyl phthalate, ethyl benzoate, trimethyl 1,2,4-benzentricarboxylate, glyme, dimethoxybenzene and combinations thereof. As used herein, the term “cresols” means a blend of the o-cresol, p-cresol and m-cresol isomers.

The non-oxygenated solvent is selected from the group consisting of xylenes, m-xylene, p-xylene, o-xylene, benzene, toluene, hexane, heptane, octane, nonane, undecane, dodecane, tridecane, tetradecane, pentadecane, hexadecane, cyclohexane, cycloheptane, cyclooctane, linear C₆ to C₁₆ alkanes, cyclic C₆ to C₁₆ alkanes and combinations thereof. As used herein, the term “xylenes” refers to a blend of xylene isomers m-xylene, p-xylene and o-xylene. In a specific example, the oxygenated solvent is cresols and the non-oxygenated solvent is xylenes.

### Generating Monoalkyl Ether

Contacting the reaction composition comprising the dialkyl ether with the metallosilicate catalyst results in the generation of an alkylene glycol monoalkyl ether. The metallosilicate catalyst functions to crack, or otherwise disassociate an alkyl moiety from, the dialkyl ether in a reactor thereby forming monoalkyl ether. Various monoalkyl ethers may be produced for different applications by varying Structure (I) of the dialkyl ether and/or by varying the glycol that may be present in the reaction composition.

The cracking of the dialkyl ether may take place at temperatures from 50° C. to 300° C. or from 100° C. to 200° C. In a specific example the reaction may be carried out at 150° C. In another specific example, the reaction may be carried out at 135° C. In another specific example, the reaction may be carried out at a temperature of 120° C. to 150° C. Cracking of the dialkyl ether may be carried out in a batch reactor, continuous reactor, or fixed-bed reactor. In operation of the chemical reaction, the Brønsted acid sites of the metallosilicate catalyst catalyze the removal of an alkyl moiety from the dialkyl ether to produce the monoalkyl ether.

The reaction which cracks the dialkyl ether and thereby generates the monoalkyl ether may be run for a time period of 0.1 hours to 10 hours. For example, the reaction may be run for 0.5 hours or greater, or 1.0 hours or greater, or 1.5 hours or greater, or 2.0 hours or greater, or 2.5 hours or greater, or 3.0 hours or greater, or 3.5 hours or greater, or 4.0 hours or greater, or 5.5 hours or greater, or 6.0 hours or greater, or 6.5 hours or greater, or 7.0 hours or greater, or 7.5 hours or greater, or 8.0 hours or greater, or 8.5 hours or greater, or 9.0 hours or greater, or 9.5 hours or greater, or 10.0 hours or greater.

The step of generating monoalkyl ether may result in a 25% or greater conversion of dialkyl ether to monoalkyl ether of. For example, the reaction may result in the conversion of 25% or greater, or 30% or greater, or 35% or greater, or 40% or greater, or 45% or greater, or 50% or greater, or 55% or greater, or 60% or greater, or 65% or greater, or 70% or greater, or 75% or greater, or 80% or greater, or 85% or greater, or 90% or greater, or 95% or greater of the dialkyl ether into monoalkyl ether. The dialkyl ether conversion is calculated as described in greater detail below.

An alkylene glycol monoalkyl ether yield of the reaction may be 10% or greater, or 15% or greater, or 20% or greater, or 25% or greater, or 30% or greater, or 35% or greater, or 40% or greater, or 45% or greater, or 50% or greater, or 55% or greater, or 60% or greater, or 65% or greater, or 70% or greater, or 75% or greater, or 80% or greater, or 85% or greater, or 90% or greater, or 95% or greater, while at the same time, 99% or less, or 95% or less, or 90% or less, or 85% or less, or 80% or less, or 75% or less, or 70% or less, or 65% or less, or 60% or less, or 55% or less, or 50% or less, or 45% or less, or 40% or less, or 35% or less, or 30% or less, or 25% or less, or 20% or less, or 15% or less. Monoalkyl ether yield is a measure of the catalytic activity and selectivity and is a good measure of the production rate of the metallosilicate catalyst. The monoalkyl ether yield is calculated as described in greater detail below.

### Examples

### Materials

Below is a listing of the materials used in the formation of the inventive examples (“IE”) and the comparative examples (“CE”).

Catalyst1 is a metallosilicate catalysts defined by a BEA structure and having a silica to alumina molar ratio of 25:1 and a surface area of 680 m²/g, that is commercially available as CP814E from ZEOLYST INTERNATIONAL™ of Conshohocken, PA.

Catalyst2 is a metallosilicate catalysts defined by a BEA structure and having a silica to alumina molar ratio of 80:1 and a surface area of 780 m²/g, that is commercially available as CBV 780 from ZEOLYST INTERNATIONAL™ of Conshohocken, PA.

Monoethylene Glycol is polyester grade with a CAS Number of 107-21-1 and is sourced from The Dow Chemical Company, Midland, Michigan.

Cresol is a mixture of O-cresol, P-cresol and M-cresol isomers in up to 8 wt % phenol and is available from SIGMA ALDRICH™ St. Louis, Missouri.

Xylene is a mixture of o-xylene, m-xylene, and p-xylene with ethylbenzene as a cosolvent and is available from SIGMA ALDRICH™ St. Louis, Missouri.

Diglyme is bis(2-methoxyethyl) ether having a CAS number of 111-96-6 and is commercially available from SIGMA ALDRICH™ St. Louis, Missouri.

Sulfated ZrO₂ was obtained from Luxfer MEL Technologies, Manchester, United Kingdom. The sulfated ZrO₂ was calcined in a box oven under static air conditions at 550° C. for 8 hours before usage.

Phosphotungstic Acid is phosphotungstic acid hydrate and was obtained from ThermoFisher Scientific (lot #: A0417659), Waltham, Massachusetts. The sample was used without any pre-treatment.

Tungstated ZrO₂ was obtained from Luxfer MEL Technologies, Manchester, United Kingdom. The sample was calcined in a box oven under static air conditions at 550° C. for 8 hours before usage.

Niobia is niobium oxides and was obtained from CBMM (lot #17162), Araxa, Brazil. The sample was calcined in a box oven under static air conditions at 400 C for 8 h before usage.

Diether (DE) is dialkyl ether having Structure (I). The dialkyl ether was produced according to the following the dialkyl ether experimental procedure.

### Dialkyl Ether Experimental Procedure

A 300 mL Parr reactor with a heating jacket and controller was used for the etherification of 1-dodecene (NEODENE 12 from Shell) and ethylene glycol with zeolite catalysts (Si/Al ratio is 25). To ensure good mixing, a pitch blade impeller was used for agitation.

The reaction mixture of 100.0 g ethylene glycol (99%, Sigma Aldrich) and 100.0 g 1-dodecene (achieve from Shell NEODENE 12) was prepared and loaded in the reactor together with 10.0-15.0 g catalyst zeolite BEA (SiO₂/Al₂O₃≈25 molar ratio, in powder form, Zeolyst) under ambient temperature. The impeller stirring rate was set to be at least 600 rpm. The reactor was heated up to 135° C. in 30 minutes, held at 135° C. for 3-6 hours and then the reactor was cooled down to room temperature by shutting off the heater. The reaction mixture was separated by the separation funnel. Next steps of draining the bottom monoethylene glycol phase with catalyst and collecting the top olefin phase were performed. Multiple batches were generated and olefin phase was collected for a distillation.

A distillation apparatus was constructed implementing a 1 L round bottom flask connected to short path head with a thermometer adapter and a condenser with a vacuum adapter at the outlet. The distillation flask was heated in an aluminum block controlled by an IKA heated stir-plate. The distillation pot was charged with the products in the olefin phase from the multiple batch reactor runs and then stirring and vacuum were applied. Significant boiling was observed but no condensate was observed or collected. The temperature of the heating block was raised to 75° C. and unreacted dodecane was collected at a distillation head temperature of 25-50° C. and a pressure of 300-100 mtorr. The heating block temperature was raised gradually to 140° C. and an intermediate fraction containing both monoether and dodecenes was recovered while the head temperature increased from 50° C. to 75° C. at a pressure of 100-90 mtorr. The monoether was collected at a head temperature of 70-115° C. and a pressure of 250-45 mtorr. The heating block temperature was raised gradually to 200° C. and an intermediate fraction containing both monoether and diether was collected while the head temperature increased from 115° C. to 130° C. at a pressure of 45-40 mtorr. The distillation was discontinued and the diether, which remained in the pot, was collected. Diether was characterized by ¹³C NMR with 97.2 wt % purity.

### Test Methods

### Gas Chromatography Samples

Prepare gas chromatography samples by mixing 100 μL of the example with 10 mL of gas chromatography solution that was prepared by addition of 1 mL of hexadecane in 1 L of ethyl acetate. Analyze the sample using an Agilent 7890B gas chromatography instrument. Determine the total amount of 1-dodecene derived species, which includes monoalkyl ether, dialkyl ether and 2-dodecanol, total amount of dodecene, which includes 1-dodecene and all non 1-dodecene other C₁₂ isomers. Table 1 provides the relevant gas chromatography instrument parameters.

TABLE 1

| Chromatograph: | Agilent 7890 Series GC | |
|---|---|---|
| Column: | Agilent HP88, 100 m × 0.25 mm × 0.20 um | |
| Detector | FID | |
| Oven: | 50° C. - 7 min - 6° C./min - 260° C. - 1 min | |
| Injector: | 250° | C. |
| Detector: | 300° | C. |
| Carrier: | Helium 2.0 mL/min constant flow mode | |
| Split ratio: | 10 | |
| Make-Up: | Nitrogen 25 mL/min | |
| Air: | 400 | mL/min |
| Hydrogen: | 40 | mL/min |
| Inlet Liner: | Restek PN 23305.5 Sky Precision Liner with wool | |
| Sample Size: | 1 | μL |

| | |
|---|---|
| GC vial rinsing | ethyl acetate |
| solvent: | |

### Dialkyl Ether (“DE”) Conversion

Dialkyl ether conversion is calculated using equation 1

$$\frac{\begin{matrix}( Pre - reaction DE concentration in RC ) - \\ ( Post - reaction DE concentration in RC )\end{matrix}}{Pre - reaction DE Concentration in RC} * 100 \% \qquad eq . 1$$

wherein “RC” is the reaction composition.

### Monoalkyl Ether (“ME”) Yield

Monoalkyl ether conversion is calculated using equation 2

$$\frac{( \frac{Post - reaction ME concentration in RC}{230.39 g / mol} )}{( \frac{Pre - reaction DE concentration in RC}{398.6 g / mol} )} * 100 \% \qquad eq . 2$$

wherein “RC” is the reaction composition, 230.09 g/mol is the molecular wight of dodecyl-monoalkyl ether, and 398.6 g/mol is the molecular wight of dodecyl-dialkyl ether.

### Sample Preparation and Results

With respect to CE1-CE5, the cracking reactions were carried out in a vial reactor. To a glass vial, 0.75 g catalyst, 6.5 g diether and 13 g ethylene glycol were added. The vial reactor was a 40 mL vial that had the reactants added along with a tumbling disc stirrer. The vial reactors were capped then placed on a multiple well hot plate to heat the vials to 135° C. as well as produce stirring from the magnetic tumbling disk stirrer. The vials were kept at temperature for 5 hours. Table 1 provide the results of CE1-CE5.

TABLE 1

| | | DE Conversion | ME Yield |
|---|---|---|---|
| Example | Catalyst | (%) | (%) |
| CE1 | Sulfated ZrO2 | 24 | 0 |
| CE2 | Phosphotungstic Acid | 59 | 0 |
| CE3 | Tungstated ZrO2 | 6 | 0.4 |
| CE4 | Niobia | 49 | 0.4 |

CE1-CE4 demonstrate that the heterogenous catalysts used did not work to effectively convert dialkyl ether to monoalkyl ether as evidence by the very low monoalkyl ether yield. While the dialkyl ether has significant conversion, the dialkyl ether was decomposed into a variety of other byproducts.

With respect to IE1-IE8, the cracking reactions were carried out in a vial reactor. To a glass vial, 0.75 g catalyst, 6.5 g diether and a varied amount of ethylene glycol were added. For IE4, 6.5 g of ethylene glycol was used. For examples IE5-IE8, 84.5 g of ethylene glycol were used. The vial reactor a 40 mL vial that had the reactants added along with a tumbling disc stirrer. The vial reactors were capped then placed on a multiple well hot plate to heat the vials to 135° C. as well as produce stirring from the magnetic tumbling disk stirrer. The vials were kept at temperature for the indicated time period. Table 2 provides the results of IE1-IE8.

TABLE 2

| | | | | DE | Monoether |
|---|---|---|---|---|---|
| | | Ethylene | Time | Conversion | Yield |
| Example | Catalyst | Glycol/DE | (h) | (%) | (%) |
| IE1 | Catalyst 1 | 0 | 1 | 64 | 26.2 |
| IE2 | Catalyst 1 | 0 | 3 | 74 | 31.6 |
| IE3 | Catalyst 1 | 0 | 5 | 75 | 31.4 |
| IE4 | Catalyst 1 | 1 | 5 | 61 | 47.3 |
| IE5 | Catalyst 1 | 13 | 1 | 59 | 27.8 |
| IE6 | Catalyst 1 | 13 | 3 | 63 | 44.4 |
| IE7 | Catalyst 1 | 13 | 5 | 71 | 31.2 |
| IE8 | Catalyst 2 | 13 | 5 | 41 | 22.2 |

Error! Reference source not found. With respect to Table 2, IE1-IE3 demonstrate Beta zeolite works well to convert dialkyl ether to monoalkyl ether in the absence of a glycol. The dialkyl ether conversion can reach 64% to 75% over 1 to 5 hours and monoalkyl ether yield can be 25% to 30%. IE4-IE8 demonstrate the addition of ethylene glycol results in improved monoether yield. Example 8 demonstrates zeolite Y is also capable of cracking diether with appreciable monoalkyl ether yield

With respect to IE9-IE15 and CE6, the cracking reactions were carried out in a vial reactor. To a glass vial, 0.75 g of catalyst 1, 1.5 g of dialkyl ether, and a balance of ethylene glycol and solvent were added. For each example, the sum of the dialkyl ether, ethylene glycol and solvent was 15 g. Each vial reactor was a 40 mL vial that had the reactants added along with a tumbling disc stirrer. The vial reactors were capped then placed on a multiple well hot plate to heat the vials to 135° C. as well as produce stirring from the magnetic tumbling disk stirrer. The vials were kept at temperature for the indicated time period. Table 3 provides the results of IE9-IE15 and CE5.

TABLE 3

| | | | | | DE | |
|---|---|---|---|---|---|---|
| | | Solvent | Ethylene | Time | Conversion | Monoether |
| Example | Solvent | Composition | Glycol/DE | (h) | (%) | Yield (%) |
| IE9 | Cresol/Xylene | 50/50 | 0 | 5 | 100 | 5.2 |
| IE10 | Cresol/Xylene | 50/50 | 1 | 5 | 100 | 17.4 |
| IE11 | Cresol/Xylene | 50/50 | 13 | 5 | 100 | 81.4 |
| IE12 | Cresol/Xylene | 50/50 | 20 | 5 | 100 | 83 |
| IE13 | Cresol | 100 | 0 | 1 | 100 | 8.6 |
| IE14 | Cresol | 100 | 13 | 1 | 100 | 58.8 |
| CE5 | Diglyme | 100 | 0 | 5 | 100 | 0 |
| IE15 | Diglyme | 100 | 13 | 5 | 100 | 64 |

With respect to Table 3, IE9-IE15 and CE5 demonstrate diether cracking in the presence of solvent and optionally ethylene glycol. As can be seen, the monoether yield is improved with the addition of ethylene glycol. The addition of ethylene glycol improves monoalkyl ether yields in all solvent systems. The addition of solvent improves monoether yield versus systems that do not use solvent.

What is claimed is:

1. A method of generating an alkylene glycol monoalkyl ether, comprising the steps of:
contacting a metallosilicate catalyst with a reaction composition comprising 5 wt % or greater of alkylene glycol dialkyl ether based on the total weight of the reaction composition; and
generating the alkylene glycol monoalkyl ether from the alkylene glycol dialkyl ether of the reaction composition.

2. The method of claim 1, wherein the metallosilicate catalyst is an aluminosilicate compound having a crystal lattice that has had one or more metal elements substituted in the crystal lattice for a silicon atom.

3. The method of claim 1, wherein the reaction composition comprises 50 wt % or greater of the alkylene glycol dialkyl ether based on the total weight of the reaction composition.

4. The method of claim 3, wherein the reaction composition comprises 80 wt % or greater of the alkylene glycol dialkyl ether based on the total weight of the reaction composition.

5. The method of claim 1, wherein the reaction composition comprises a glycol selected from the group consisting of monoethylene glycol, diethylene glycol, glycerol or combinations thereof.

6. The method of claim 5, wherein the reaction composition comprises from 10 wt % to 80 wt % of the glycol based on the total weight of the reaction composition.

7. The method of claim 1, wherein the reaction composition further comprises a solvent selected from the group consisting of an oxygenated solvent, a non-oxygenated solvent and combinations thereof.

8. The method of claim 7, wherein the solvent is from 1 wt % to 80 wt % of the total weight of the reaction composition.

9. The method of claim 1, wherein the solvent comprises both the oxygenated solvent and the non-oxygenated solvent.

10. The method of claim 9, wherein the solvent comprises from 40 wt % to 60 wt % of the non-oxygenated solvent based on a total weight of the solvent.
